(19) United States
(12) Patent Application Publication
(10) Pub. No.: US 2014/0128858 A1
(43) Pub. Date: May 8, 2014

(54) SYSTEMS AND METHODS FOR PERFORMING ENDOMETRIAL ABLATION

(71) Applicant: COVIDIEN LP, Mansfield, MA (US)

(72) Inventor: JAMES H. ORSZULAK, NEDERLAND, CO (US)

(73) Assignee: COVIDIEN LP, Mansfield, MA (US)

(21) Appl. No.: 14/064,409

(22) Filed: Oct. 28, 2013

Related U.S. Application Data
(60) Provisional application No. 61/724,171, filed on Nov. 8, 2012.

(51) Int. Cl. A61F 7/00

(52) U.S. Cl. CPC A61F 7/007
USPC 606/27

(57) ABSTRACT

A system for transmitting electrosurgical energy from a generator to an electrosurgical instrument for electro-thermal ablation is disclosed. The generator includes one or more active output terminals which supply energy to tissue and are operatively connected to one or more supply lines. The generator also includes one or more return output terminals which return energy from the tissue and are operatively connected to one or more return lines. The system also includes an electrosurgical cable housing a portion of the one or more supply lines and one or more return lines. The electrosurgical instrument includes a helical electro-thermal element operatively connected to the one or more supply lines and the one or more return lines. The one or more supply lines and one or more return lines are wound in a double helix arrangement such that the electrical field along the cable and helical electro-thermal element is mitigated along the lengths thereof.

### CROSS REFERENCE TO RELATED APPLICATION

The present application claims the benefit of and priority to U.S. Provisional Application Ser. No. 61/724,171, filed on Nov. 8, 2012, the entire contents of which are incorporated herein by reference.

### BACKGROUND

1. Technical Field

The present disclosure relates to devices suitable for tissue ablation applications and, more particularly, to systems and methods for performing endometrial ablation without contacting the endometrium.

2. Discussion of Related Art

Abnormal vascular bleeding of the endometrium tissue of the uterus is generally treated by using a laser beam (laser thermal ablation) or by applying heat to the endometrium tissue. These methods of treatment, referred to as endometrial ablation, destroy (ablate) the uterine lining, or endometrium. The endometrium heals by scarring, which usually reduces or prevents uterine bleeding. A lighted viewing instrument or hysteroscope may be used to see inside the uterus during endometrial ablation.

Several contact methods of applying heat or thermal energy to the endometrium tissue during endometrial ablation include filling a balloon with saline solution that has been heated to 85 degrees Celsius (thermal balloon ablation), and applying RF or microwave energy to the endometrium tissue. The latter methods typically include contacting the uterine tissue with electrodes or antennas capable of transmitting RF and microwave energy to the endometrium tissue. These conventional contact methods for ablating the endometrium tissue can cause accidental puncture (perforation) of the uterus, burns (thermal injury) to the uterus, and electrical shock due to fluids and contact electrode mesh systems.

For example, one RF energy contact procedure uses a deployed metal electrode array mesh using electrical RF ablation energy to desiccate the endometrium tissue by direct contact. Although this procedure only typically takes 90 seconds, direct contact of the RF energy presents a leakage hazard to the patient if not properly controlled. Other contact techniques, such as using a microwave wand for applying microwave energy, have been identified which potentially create irregular ablation of the endometrium, resulting in a less than optimal clinical effect.

Other contact methods, such as thermal balloon ablation, have been shown to create scar tissue which can shut the cervix, requiring a procedure to be performed to release the blood collected in the uterus. This is due to the fact that heated fluids retain thermal energy which can overdose tissue treatment due to stored thermal energy latency.

Another conventional contact technique used for endometrial ablation uses a cryo ablation freezing method, where the cooling thermal energy is applied for 10 to 20 minutes, resulting in the uterine lining to fall off. Thermal cooling also presents a thermal energy latency which can result in excessive tissue damage and less than optimal clinical efficacy.

Therefore, non-contact systems and methods are desired which overcome the disadvantages and minimize or eliminate potential hazards to the patient as compared to conventional contact endometrial ablation procedures.

### SUMMARY

Various embodiments of the present disclosure provide non-contact systems and methods for performing endometrial ablation. As used herein, the term “distal” refers to the portion that is being described which is further from a user, while the term “proximal” refers to the portion that is being described which is closer to a user. Further, to the extent consistent with one another, any of the aspects described herein may be used in conjunction with any of the other aspects described herein.

The term “energy-delivery device” is defined herein to include any surgical instrument, device or apparatus, such as the helical electro-thermal heating element described herein, capable of delivering energy to tissue, such as the endometrium, including, but not limited to, radiofrequency and microwave energy.

In accordance with aspects of the present disclosure, electro-thermal ablation systems are provided generally including at least one energy-delivery device for delivering energy to tissue, such as the endometrium, without contacting the endometrium. The energy-delivery device as described herein and shown by the various figures is a helical electro-thermal element housed within an electrosurgical instrument and operatively connected to one or more supply and return lines.

The helical electro-thermal element converts RF energy to thermal energy which is transferred by thermal conductive material to the endometrium for performing endometrial ablation of the uterine tissue without contacting the endometrium. The helical electro-thermal element device avoids potential shock hazard as compared to conventional fluid- or vapor-based ablation devices, including direct contact ablation systems, such as direct contact electrode mesh systems.

In particular, the helical electro-thermal element delivers RF or other types of energy, such as microwave energy, to a thermally conductive material placed within the uterus which transfers the therapeutic heat energy to the endometrium tissue. The heat energy is transferred to the endometrium tissue without the electro-thermal element making contact with the endometrium tissue.

The thermally conductive material can include foams or composite elastomeric materials capable of transferring heat energy with minimal thermal storage latency to avoid excessive thermal overdose treatment. The thermally conductive material transfers the heat energy and ablates the endometrium uniformly to provide greater control of therapeutic treatment.

The helix configuration of the helical electro-thermal element according to the present disclosure provides a safe and effective treatment method for delivering RF or other types of thermal energy to the endometrium via the thermally conductive material by minimizing field leakage and thereby preventing electrical shock. Other advantages of using a helical energy transmission medium are discussed in commonly-owned U.S. Pat. No. 7,819,865, the entire contents of which are incorporated herein by reference.

According to one aspect of the present disclosure, there is provided an electrosurgical system including a generator, a cable housing one or more supply lines configured for transmitting energy and one or more return lines, and an energy-delivery device operatively connected to the one or more supply lines and the one or more return lines. The energy-delivery device is adapted to direct energy to tissue without contacting the tissue. The one or more supply lines and the one or more return lines are arranged along a length of the energy-delivery device in a helical arrangement with respect to each other. The electrosurgical system further includes a handle assembly. The energy-delivery device is actuated by the handle assembly.

The energy-delivery device is a helical electro-thermal heating element which is housed by an elongated shaft. The elongated shaft further houses a thermally conductive material, such as foam or a composite elastomeric material.

The electrosurgical system further includes at least one sensor in proximity to the energy-delivery device for sensing at least one operating parameter or tissue parameter. A signal indicative of the at least one operating parameter or tissue parameter is transmitted from the at least one sensor to the generator via a conductive or fiber optic cable. The one or more supply lines and the one or more return lines are arranged along a length of the cable in a helical arrangement with respect to each other.

According to another aspect of the present disclosure, there is provided an electrosurgical system including a generator adapted to generate electrosurgical energy for treating tissue. The generator includes one or more active output terminals which supply energy to the tissue. The active output terminals are operatively connected to one or more supply lines. The generator also includes one or more return output terminals which are configured to return energy from the tissue. The return output terminals being operatively connected to one or more return lines. The electrosurgical system further includes an electrosurgical cable housing a portion of the one or more supply lines and one or more return lines, and an electrosurgical instrument operatively connected to the electrosurgical cable. The electrosurgical instrument includes a helical electro-thermal element operatively connected to the one or more supply lines and the one or more return lines. The helical electro-thermal element is adapted for delivering thermal energy to tissue without contacting the tissue. The one or more supply lines and the one or more return lines are wound in a double helix arrangement such that the electrical field along the cable and helical electro-thermal element is mitigated along the lengths thereof.

The electrosurgical system further includes a handle assembly. The energy-delivery device is actuated by said handle assembly. The electrosurgical instrument includes an elongated shaft housing the helical electro-thermal element and a thermally conductive material, such as foam or a composite elastomeric material.

The electrosurgical system further includes at least one sensor in proximity to the helical electro-thermal element for sensing at least one operating parameter or tissue parameter. A signal indicative of the at least one operating parameter or tissue parameter is transmitted from the at least one sensor to the generator via a conductive or fiber optic cable. The one or more supply lines and the one or more return lines are arranged along a length of the cable in a helical arrangement with respect to each other.

According to another aspect of the present disclosure, a method for performing endometrial ablation is provided. The method includes providing a generator adapted to generate electrosurgical energy; delivering the electrosurgical energy to an electrosurgical instrument having an electro-thermal element configured for converting the electrosurgical energy to thermal energy; and transferring the thermal energy to the endometrium via a thermally conductive material disposed within the uterus. The thermal energy ablates the endometrium without the electrosurgical instrument making contact with the endometrium. The electrosurgical instrument is operatively connected to the generator via a cable housing one or more supply lines and one or more return lines. The one or more supply lines and the one or more return lines are wound in a double helix arrangement such that the electrical field along the cable is mitigated along the length thereof.

The method further includes sensing at least one parameter and transmitting a signal to the generator indicative of the at least one sensed parameter. The thermally conductive material is a foam or an elastomeric composite material.

### BRIEF DESCRIPTION OF THE DRAWINGS

Objects and features of the presently-disclosed systems and methods for performing endometrial ablation using an electrosurgical instrument having a helical electro-thermal element will become apparent to those of ordinary skill in the art when descriptions of various embodiments thereof are read with reference to the accompanying drawings, of which:

FIG. 1 is a schematic diagram of an electrosurgical system for delivering thermal energy to tissue, such as the endometrium, via a helical electro-thermal element housed within an electrosurgical instrument for performing endometrial ablation in accordance with an embodiment of the present disclosure;

FIG. 2 is a schematic diagram of a laparoscopic shaft shrouding the helical electro-thermal element in accordance with an embodiment of the present disclosure;

FIG. 3 is a schematic illustration of the helical electro-thermal element delivering thermal energy to the endometrium in accordance with an embodiment of the present disclosure; and

FIG. 4 is a schematic illustration of an electrosurgical instrument having a pivotable laparoscopic shaft in accordance with an embodiment of the present disclosure.

### DETAILED DESCRIPTION

Particular embodiments of the present disclosure are described hereinbelow with reference to the accompanying drawings. In the following description, well-known functions or constructions are not described in detail to avoid obscuring the present disclosure in unnecessary detail. Those skilled in the art will understand that the invention according to the present disclosure may be adapted for use with either monopolar or bipolar electrosurgical systems and either an endoscopic instrument or an open instrument. It should also be appreciated that different electrical and mechanical connections and other considerations apply to each particular type of instrument.

This description may use the phrases “in an embodiment,” “in embodiments,” “in some embodiments,” or “in other embodiments,” which may each refer to one or more of the same or different embodiments in accordance with the present disclosure. For the purposes of this description, a phrase in the form “NB” means A or B. For the purposes of the description, a phrase in the form “A and/or B” means “(A), (B), or (A and B)”. For the purposes of this description, a phrase in the form “at least one of A, B, or C” means “(A), (B), (C), (A and B), (A and C), (B and C), or (A, B and C)”.

The present disclosure provides for an electrosurgical transmission cable wound in a double helix having a proximal geometric relationship in three-dimensional physical space, to control the inductive and capacitive components of the transmission cable and significantly reduce the capacitive leakage due to RF radiation. The transmission cable according to present disclosure is wound in a double helix to minimize the stray RF radiation by reducing the transmitting antenna effect for transmission mediums shorter than ½ wavelength. A distal portion of the cable extends within a laparoscopic shaft of an electrosurgical instrument and defines an electro-thermal element configured to deliver thermal energy to tissue via thermally conductive material surrounding the electro-thermal element.

In particular, in accordance with the present disclosure, electrosurgical systems and methods are provided for transmitting electrosurgical energy from a generator to an electrosurgical instrument. The systems and methods are configured for treating tissue, such as the endometrium, by delivering thermal energy to tissue and without contacting the tissue. The generator includes one or more active output terminals which deliver the energy generated by the generator to the electrosurgical instrument via one or more supply lines of a cable. The energy delivered to the electrosurgical instrument can include, for example, RF or microwave energy, or both. The generator also includes one or more return output terminals which return energy from the tissue. The return output terminals are operatively connected to one or more return lines of the cable.

The electrosurgical instrument includes an energy-delivery device for delivering energy to tissue for electro-thermal ablation without contacting the tissue. The energy-delivery device can be a helical electro-thermal element operatively connected to the one or more supply lines and one or more return lines of the cable and housed within a shaft of the electrosurgical instrument. The one or more supply lines and one or more return lines are wound in a double helix arrangement such that the electrical field along the cable and electro-thermal element is mitigated.

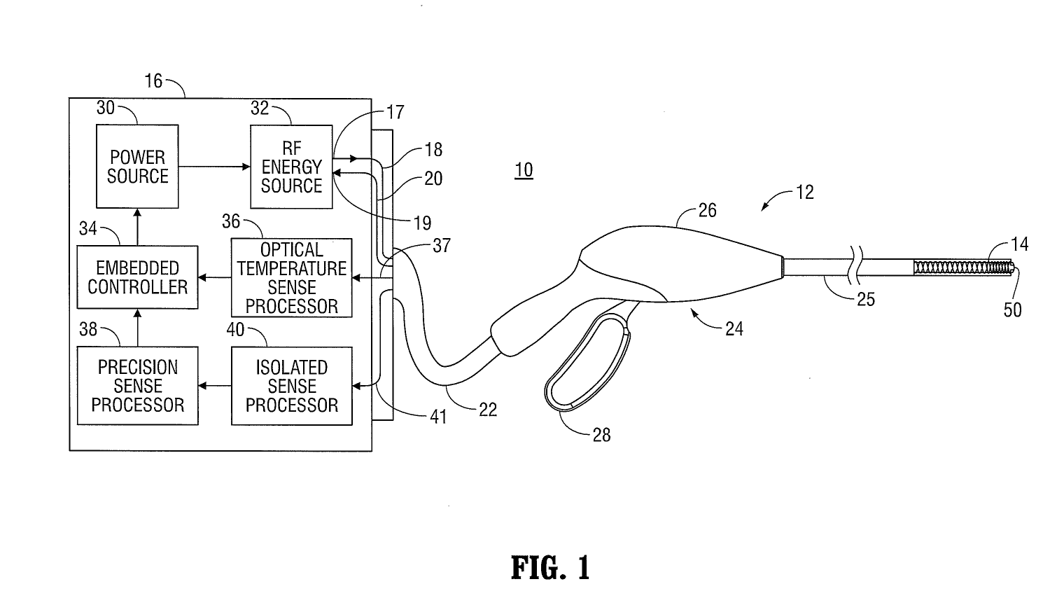

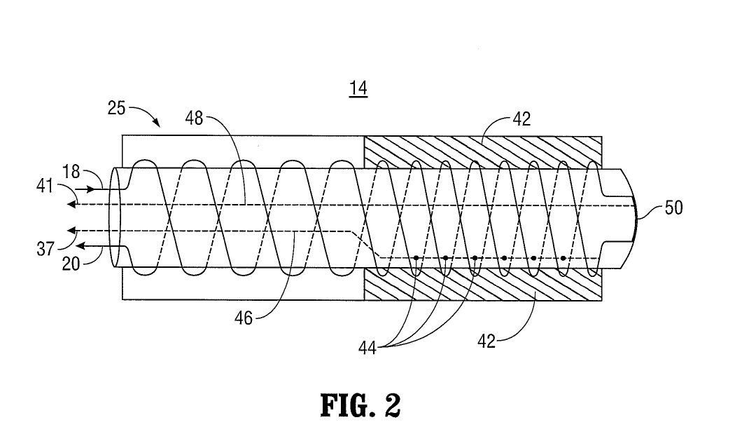

FIG. 1 shows an electrosurgical system 10 according to the present disclosure for delivering RF-based energy through a helix cable 22 to an electrosurgical instrument 12 containing a helical electro-thermal element 14 for endometrial ablation thermal treatment without contacting the endometrium. The helical electro-thermal element 14 is shown in greater detail in FIG. 2.

The helical electro-thermal element 14 is connected to an energy source (e.g., a generator 16) that communicates electrosurgical RF-based energy to the electrosurgical instrument 12 during a treatment operation of the system 10. Electrosurgical RF-based energy or other forms of energy is supplied to the helical electro-thermal element 14 by generator 16 via one or more supply or active lines 18 and returned through the one or more return lines 20. The supply and return lines 18, 20 or conductive traces deliver the prescribed energy through the helix configured cable geometry to minimize the electric V/m and electromagnetic Nm fields enroute to the helical electro-thermal element 14. The conductive traces 18, 20 are partially enclosed or housed within cable 22, handle assembly 24 and flexible laparoscopic shaft 25 of electrosurgical instrument 12, and generator 16.

Handle assembly 24 includes a fixed handle 26 and a movable handle 28. Handle 28 moves relative to the fixed handle 26 to trigger the treatment operation by activating generator 16 for generating and delivering energy to the electrosurgical instrument 12. Flexible laparoscopic shaft 25 shrouds the helical electro-thermal element 14 and thermal material 42.

A portion of supply and return lines 18, 20 or conductive traces define the helical electro-thermal element 14 as shown by FIG. 2. The conductive traces provide low loss energy transmission to the electro-thermal element 14 which converts electrical energy to thermal energy for vascular treatment. The electro-thermal conductors or supply and return lines 18, 20 of the electro-thermal element 14 represent a continuous extension of the conductive traces 18, 20, but have increased resistivity and a patterned geometry to generate thermal heat energy. In this configuration, energy is sourced from the generator 16 as required for delivery to the electro-thermal heat element 14, where the energy is transferred to thermal material 42 surrounding the electro-thermal element 14 and closed loop control monitored through signal feedback via helix cable communication portal.

With reference to FIG. 1, the generator 16 includes suitable input controls (e.g., buttons, activators, switches, touch screen, etc.) for controlling the generator 16. In addition, the generator 16 may include one or more suitable display screens for providing the operator with variety of output information (e.g., intensity settings, treatment complete indicators, etc.). The controls allow the operator to adjust power of the energy generated, adjust the waveform, and other suitable parameters to achieve the desired operating characteristics and specifications suitable for a particular task (e.g., endometrial ablation). The electrosurgical instrument 12 may also include a plurality of input controls that may be redundant with certain input controls of the generator 16. Placing the input controls at the instrument 12 allows for easier and faster modification of energy parameters during the treatment procedure (e.g., endometrial ablation) without requiring interaction with the generator 16.

FIG. 1 also includes a block diagram of the generator 16 having power source 30, RF energy source 32 with one or more active output terminals 17 and one or more return terminals 19, embedded controller 34, and the following sense processors: optical temperature sense processor 36, precision sense processor 38, and isolated sense processor 40. The generator 16 and its various components are known to one or ordinary skill in the art.

In particular, the power source 30 provides power to the generator 16. The RF energy source 32 generates RF energy for transmission to the electrosurgical instrument 12 via active output terminal 17. The embedded controller 34 is programmed to perform the various functions for controlling the generator including actuating the generator for generating RF energy during a treatment operation.

The optical temperature sense processor 36 receives a signal 37 transmitted thereto via fiber optic cable 46. The signal 37 is generated using fiber Bragg or Fabry Perot temperature sense optical gratings 44 which monitor the delivered heat energy and treatment temperature in real-time.

The isolated sense processor 40 provides a conversion of the sensed RF energy parameters communicated as a signal 41 in real-time from energy sensor 50 via conductor 48 through an isolation barrier.

The precision sense processor 38 receives and processes the energy sensory signals from the isolated sense processor 40, to delineate functional control of the necessary sensory control parameters received from the treatment site. As a minimum but not limited to, the precision sense processor monitors and computes the RF current, voltage, power and differential energy parameter time rates of change, which are closed loop control processed by the embedded controller 34.

A fiber optic pressure sensor can also be utilized to monitor any puncture of the endometrium.

Cable 22 provides a transmission medium to deliver RF energy from the generator 16 to a tissue site. The cable 22 represents one example of a preferred embodiment for the RF transmission medium, which reduces the radiated RF electrical field and maximizes the applied clinical treatment energy delivered to the tissue site.

The cable 22 according to the present disclosure orients the supply and return lines 18, 20 so that the electrical fields generated therethrough are canceled, thereby reducing the amount of leaked stray RF energy. More specifically, placement and orientation of the lines 18, 20 in the manner discussed above provides for close proximity of electrical fields generated during transmission of electrosurgical RF energy and maximizes amount of energy delivered to the treatment site. Reducing the electrical fields also increases safety of personnel and the patient.

Reduced RF radiation decreases capacitive and RF field leakage and improves RF control of the delivered energy. Reduced RF radiation also decreases RE transmission loss and improves efficiency of the generator 16 by reducing RF harmonic component, minimizing corruption of the RF source and reducing peripheral conductive and radiative emissions. Further, reducing RF radiation also decreases the RF noise to additional equipment found in the room, such as patient monitoring equipment.

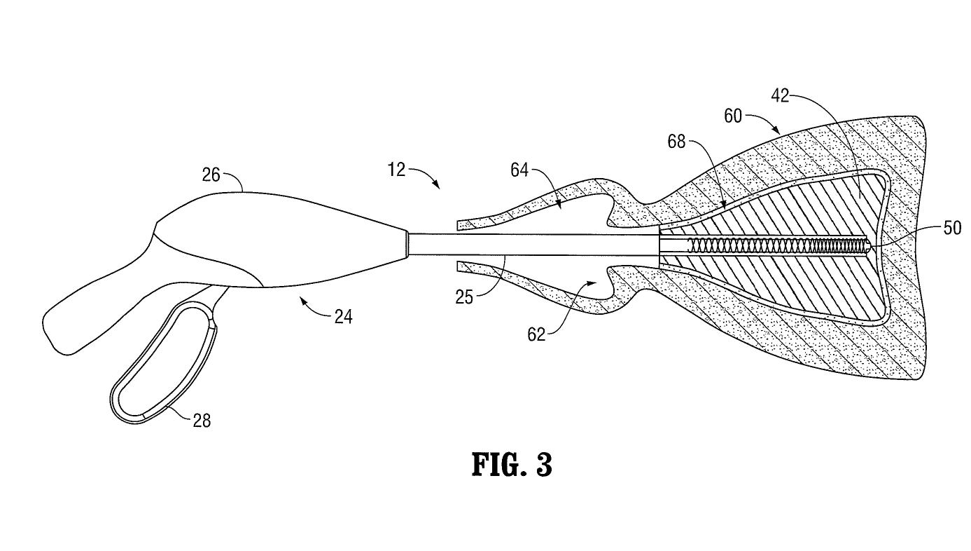

FIG. 3 illustrates the instrument 12 inserted through the vaginal canal 64 and the cervix 62 to the uterus 60. Upon entering the uterus 60, the thermal material 42 is mechanically deployed from within the shaft 25 to contact the endometrium uterine tissue 68. The thermal material 42 can be deployed by proximally moving the shaft 25 and thereby releasing the material 42 from within the shaft. The thermal material 42 fills the cavity of uterus 60 and conducts thermal energy from the electro-thermal heating element 14 to the endometrium uterine tissue 68 for ablating the tissue 68 uniformly during endometrial ablation.

The thermally conductive material 42 can include foams or composite elastomeric materials capable of transferring heat energy with minimal thermal storage latency to avoid excessive thermal overdose treatment.

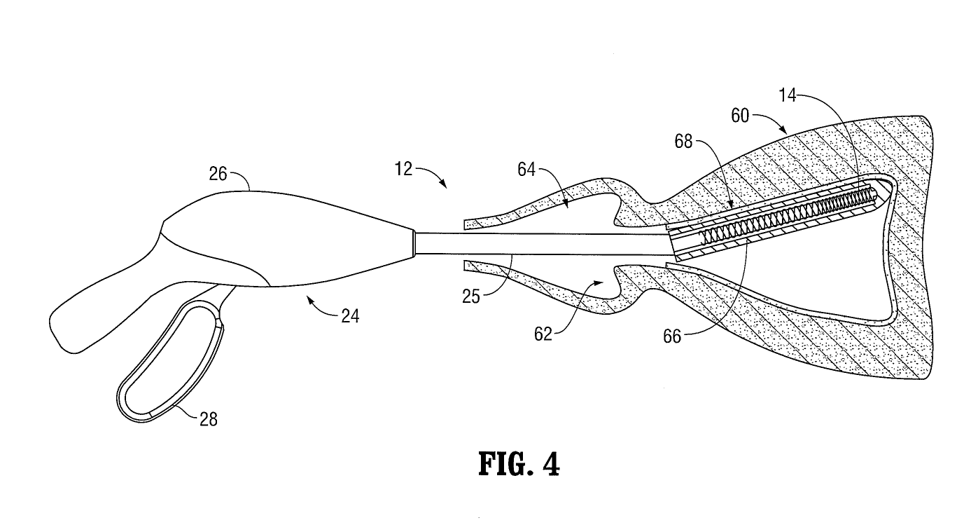

In an alternate embodiment shown by FIG. 4, a portion of the shaft 25 is pivots or articulates for enabling that portion of the shaft 25 to be positioned along the endometrium uterine tissue 68. When the shaft 25 is in proximity to the tissue 68, the thermal material 42 is deployed and the electro-thermal heating element 14 is activated for performing endometrial ablation. The thermal energy is delivered to specific vascular bleeding areas of the endometrium uterine tissue 68 from the electro-thermal heating element 14 via the thermal material 42. The electro-thermal heating element 12 in this manner follows the deployed thermal material 42 and only transfers thermal heat energy to the specific locations of the bleeding endometrium for optimal clinical treatment efficacy and minimal uterine thermal damage.

The methods of the present disclosure eliminate the retaining of heat energy within the uterus as compared with prior art methods, such as using balloons filled with heated saline. Thermally conductive foams and elastomeric composite materials dissipate thermal energy faster than saline.

Although embodiments have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

What is claimed is:

1. An electrosurgical system, comprising:
a generator;
a cable housing one or more supply lines configured for transmitting energy and one or more return lines;
an energy-delivery device operatively connected to the one or more supply lines and the one or more return lines, the energy-delivery device adapted to direct energy to tissue without contacting the tissue, wherein the one or more supply lines and the one or more return lines are arranged along a length of the energy-delivery device in a helical arrangement with respect to each other.

2. The electrosurgical system of claim 1, further comprising a handle assembly, and wherein the energy-delivery device is actuated by the handle assembly.

3. The electrosurgical system of claim 1, wherein the energy-delivery device is a helical electro-thermal heating element.

4. The electrosurgical system of claim 1, further comprising an elongated shaft housing the energy-delivery device and a thermally conductive material.

5. The electrosurgical system of claim 4, wherein the thermally conductive material is foam or a composite elastomeric material.

6. The electrosurgical system of claim 1, further comprising at least one sensor in proximity to the energy-delivery device for sensing at least one operating parameter or tissue parameter.

7. The electrosurgical system of claim 6, wherein a signal indicative of the at least one operating parameter or tissue parameter is transmitted from the at least one sensor to the generator via conductive or fiber optic cable means.

8. The electrosurgical system of claim 1, wherein the one or more supply lines and the one or more return lines are arranged along a length of the cable in a helical arrangement with respect to each other.

9. The electrosurgical system of claim 1, wherein the tissue is the endometrium.

10. An electrosurgical system comprising:
a generator adapted to generate electrosurgical energy for treating tissue, the generator including one or more active output terminals which supply energy to the tissue, the active output terminals are operatively connected to one or more supply lines, the generator also including one or more return output terminals which are configured to return energy from the tissue, the return output terminals being operatively connected to one or more return lines;
an electrosurgical cable housing a portion of the one or more supply lines and one or more return lines; and
an electrosurgical instrument operatively connected to the electrosurgical cable, the electrosurgical instrument including a helical electro-thermal element operatively connected to the one or more supply lines and the one or more return lines, wherein the helical electro-thermal element is adapted for delivering thermal energy to tissue without contacting the tissue, and wherein the one or more supply lines and the one or more return lines are wound in a double helix arrangement such that the electrical field along the cable and helical electro-thermal element is mitigated along the lengths thereof.

11. The electrosurgical system of claim 10, further comprising a handle assembly, and wherein the energy-delivery device is actuated by the handle assembly.

12. The electrosurgical system of claim 10, wherein the electrosurgical instrument comprises an elongated shaft housing the helical electro-thermal element and a thermally conductive material.

13. The electrosurgical system of claim 12, wherein the thermally conductive material is foam or a composite elastomeric material.

14. The electrosurgical system of claim 10, further comprising at least one sensor in proximity to the helical electro-thermal element for sensing at least one operating parameter or tissue parameter.

15. The electrosurgical system of claim 14 wherein a signal indicative of the at least one operating parameter or tissue parameter is transmitted from the at least one sensor to the generator via conductive or fiber optic cable means.

16. The electrosurgical system of claim 10, wherein the one or more supply lines and the one or more return lines are arranged along a length of the cable in a helical arrangement with respect to each other.

17. The electrosurgical system of claim 10, wherein the tissue is the endometrium.

18. A method for performing ablation of the endometrium comprising:
providing a generator adapted to generate electrosurgical energy;
delivering the electrosurgical energy to an electrosurgical instrument having an electro-thermal element configured for converting the electrosurgical energy to thermal energy; and
transferring the thermal energy to the endometrium via a thermally conductive material disposed within the uterus, wherein the thermal energy ablates the endometrium without said electrosurgical instrument making contact with the endometrium;
wherein the electrosurgical instrument is operatively connected to the generator via a cable housing one or more supply lines and one or more return lines, and wherein the one or more supply lines and the one or more return lines are wound in a double helix arrangement such that the electrical field along the cable is mitigated along the length thereof.

19. The method of claim 18, further comprising sensing at least one parameter and transmitting a signal to the generator via conductive or fiber optic cable means, indicative of the at least one sensed parameter.

20. The method of claim 18, wherein the thermally conductive material is a foam or a composite elastomeric material.
